(19) United States
(12) Patent Application Publication
(10) Pub. No.: US 2017/0298436 A1
(43) Pub. Date: Oct. 19, 2017

(54) GROUP TESTING APPROACH FOR A GENETIC SCREENING ASSAY

(71) Applicant: Counsyl, Inc., South San Francisco, CA (US)

(72) Inventors: Kristjan Eerik KASENIIT, San Francisco, CA (US); Mark R. THEILMANN, South San Francisco, CA (US); Alexander De Jong ROBERTSON, South San Francisco, CA (US); Eric Andrew EVANS, Brisbane, CA (US); Imran Saeedul HAQUE, San Francisco, CA (US)

(21) Appl. No.: 15/488,129

(22) Filed: Apr. 14, 2017

Related U.S. Application Data
(60) Provisional application No. 62/323,441, filed on Apr. 15, 2016.

(51) Int. Cl. C12Q 1/68; G01N 27/447; G06F 19/18; G06F 19/00; G06F 19/26

(52) U.S. Cl. CPC C12Q 1/6883; G06F 19/366; G06F 19/26; G06F 19/18; G01N 27/44791; C12Q 2600/16; C12Q 2600/156

(57) ABSTRACT

According to one aspect, systems and processes for assaying a plurality of nucleic acid samples are provided. In an exemplary process, a matrix is generated including pools and samples using a pooling scheme with decoding capability equal to a number D. Matrix organization includes assigning one pool in a set of pools per row by one sample in a set of samples per column. Sample assignment creates a known pattern of pools, wherein each sample in the set of pools is assigned a total number of D+1 times and any two pools have at most one sample in common. Samples are pooled based on a pooling scheme, where pooled samples are assayed. Positive pools are determined and one or more positive samples are identified. The matrix is displayed as a visual pattern representing the known pattern of pools, the identified positive samples, and the determined positive pools.

### CROSS-REFERENCE TO RELATED APPLICATIONS

This application claims priority to U.S. Provisional Ser. No. 62/323,441, filed on Apr. 15, 2016, entitled “A GROUP TESTING APPROACH FOR TRINUCLEOTIDE REPEAT EXPANSION DISORDER SCREENING,” and is incorporated herein by reference for all purposes.

### FIELD

The following disclosure relates generally to a group testing scheme for testing a plurality of nucleic acid samples.

### BACKGROUND

Nucleic acid repeats are associated with various diseases. For example, expansion of a CGG triplet repeat sequence in the 5′ UTR of the Fragile X mental retardation 1 (FMR1) gene (OMIM *309550) is associated with Fragile X syndrome (FXS, OMIM #300624), the most common inherited form of mental retardation. FXS testing is commonly performed in expanded carrier screening, and has been proposed for inclusion in newborn screening. Expansion of this repeat into the full mutation range (>200 repeats) triggers methylation and transcriptional silencing of FMR1, causing FXS. Alleles are categorized by their repeat count, for which in the “normal” range (5-44 repeats), the repeat sequence is stable between generations. Intermediate alleles (45-54 repeats) require at least two generations to expand to full mutations, and premutation alleles (55-200 repeats) may expand to full mutations when passed from a mother to her child. Full mutation alleles are diagnostic for FXS in males, where females are affected with <50% penetrance and show milder symptoms. Men and women with the premutation are at risk for a number of related symptoms such as ataxia or premature ovarian failure.

Due to the difficulty of amplifying long triplet repeats, traditional tests for FXS carrier status have relied on Southern blotting to detect expanded CGG repeats. Recent advances in polymerase chain reaction (PCR) methods allow detection of these repeats with accuracy and sensitivity equal to Southern blotting. Capillary electrophoresis of the PCR product makes it possible to quantify the CGG repeat count, but requires laborious peak-calling and counting. However, because pathogenic alleles are long and have low complexity (>200 CGG repeats), FXS is currently tested by a singleplex electrophoresis-resolved PCR assay rather than multiplexed approaches like next-generation sequencing (NGS) or mass spectrometry. Although long-read NGS approaches have been described, they are substantially more expensive than the PCR fragment sizing methods.

Thus, although NGS has markedly reduced the cost of many genetic tests, FXS testing has remained static in cost since the introduction of PCR-based methods. Furthermore, although utilization of existing multiplexed methods may use various pooling schemes (i.e., a Shifted Transversal Design scheme), there exists a need to develop an approach for group testing of samples which achieves improved cost savings over such existing methods. Shifted Transveral Design scheme is described, for example, in “A new pooling strategy for high-throughput screening: the Shifted Transversal Design,” Nicolas Thierry-Mieg (2006), hereby incorporated by reference in its entirety. Few approaches have explicitly considered the analytical sensitivity of assays as a limiting factor in pooling design, and further have not provided a desirable cost reduction. Therefore, in order to enable screening at scales comparable to those enabled by NGS or affordable testing, an optimized multiplexed method for screening rare diseases is desired.

### SUMMARY

According to one aspect of the present disclosure, a method for assaying a plurality of nucleic acid samples is provided, the method comprising: generating a matrix of pools and samples using a pooling scheme, wherein the pooling scheme has a decoding capability equal to a number D; organizing the matrix by assigning one pool in a set of pools per row by one sample in a set of samples per column; assigning a number R of samples to each pool to create a known pattern of pools, wherein each sample in the set of pools is assigned a total number of D+1 times and any two pools have at most one sample in common; pooling the plurality of samples based at least in part on the pooling scheme and the matrix; assaying the pooled samples; in response to the assaying, determining a number of positive pools; identifying one or more positive samples based on the determined positive pools and the known pattern of pools; and displaying, on a display screen, the matrix as a visual pattern, the visual pattern representing each of the known pattern of pools, the identified one or more positive samples, and the determined positive pools.

In some embodiments, a number of positive samples P is determined in a single iteration of analysis when P≦D. In some embodiments, the method comprises performing at least one additional round of assaying when a number of positive samples P>D. In some embodiments, when exactly P positive samples are present in the plurality of samples, determining a number of positive pools comprises: if P≦D, identifying P*(D+1) positive pools; and if P>D, determining that (i) the determined positive pools include colliding samples, and (ii) at least one additional round of assaying is required. In some embodiments, each individual pool includes a tested set of samples distinct from each other individual pool. In some embodiments, the pooling scheme is a nonadaptive pooling scheme, such that samples are arranged in overlapping pools associated with the known pattern. In some embodiments, the decoding capability D is equal to 1. In some embodiments, the matrix has a size equal to: (R+1) by (R*(R+1))/2. In some embodiments, the number D is greater than 1. In some embodiments, the number D is constrained based on a quantity of sample material available. In some embodiments, the number R is constrained by the analytical sensitivity of the assaying. In some embodiments, the pooling scheme is utilized in the detection of Fragile X Syndrome. In some embodiments, assaying the pooled samples further comprises utilization of a capillary electrophoresis assay.

In some embodiments, the present invention includes a system for assaying a plurality of nucleic acid samples, the system comprising: a display; one or more processors; and a memory storing one or more programs, wherein the one or more programs include instructions configured to be executed by the one or more processors, causing the one or more processors to perform operations comprising: generating a matrix of pools and samples using a pooling scheme, wherein the pooling scheme has a decoding capability equal to a number D; organizing the matrix by assigning one pool in a set of pools per row by one sample in a set of samples per column; assigning a number R of samples to each pool to create a known pattern of pools, wherein each sample in the set of pools is assigned a total number of D+1 times and any two pools have at most one sample in common; pooling the plurality of samples based at least in part on the pooling scheme and the matrix; assaying the pooled samples; in response to the assaying, determining a number of positive pools; identifying one or more positive samples based on the determined positive pools and the known pattern of pools; and displaying, on a display screen, the matrix as a visual pattern, the visual pattern representing each of the known pattern of pools, the identified one or more positive samples, and the determined positive pools.

In some embodiments, a number of positive samples P is determined in a single iteration of analysis when P≦D. In some embodiments, the one or more programs further include instructions for: performing at least one additional round of assaying when a number of positive samples P>D. In some embodiments, when exactly P positive samples are present in the plurality of samples, determining a number of positive pools comprises: if P≦D, identifying P*(D+1) positive pools; and if P>D, determining that (i) the determined positive pools include colliding samples, and (ii) at least one additional round of assaying is required. In some embodiments, each individual pool includes a tested set of samples distinct from each other individual pool. In some embodiments, the pooling scheme is a nonadaptive pooling scheme, such that samples are arranged in overlapping pools associated with the known pattern. In some embodiments, the decoding capability D is equal to 1. In some embodiments, the matrix has a size equal to: (R+1) by (R*(R+1))/2. In some embodiments, the number D is greater than 1. In some embodiments, the number D is constrained based on a quantity of sample material available. In some embodiments, the number R is constrained by the analytical sensitivity of the assaying. In some embodiments, the pooling scheme is utilized in the detection of Fragile X Syndrome. In some embodiments, assaying the pooled samples further comprises utilization of a capillary electrophoresis assay.

In some embodiments, the present invention includes a non-transitory computer readable storage medium having instructions stored thereon, the instructions, when executed by one or more processors, cause the processors to perform operations for assaying a plurality of nucleic acid samples, the operations comprising: generating a matrix of pools and samples using a pooling scheme, wherein the pooling scheme has a decoding capability equal to a number D; organizing the matrix by assigning one pool in a set of pools per row by one sample in a set of samples per column; assigning a number R of samples to each pool to create a known pattern of pools, wherein each sample in the set of pools is assigned a total number of D+1 times and any two pools have at most one sample in common; pooling the plurality of samples based at least in part on the pooling scheme and the matrix; assaying the pooled samples; in response to the assaying, determining a number of positive pools; identifying one or more positive samples based on the determined positive pools and the known pattern of pools; and displaying, on a display screen, the matrix as a visual pattern, the visual pattern representing each of the known pattern of pools, the identified one or more positive samples, and the determined positive pools.

In some embodiments, a number of positive samples P is determined in a single iteration of analysis when P≦D. In some embodiments, the one or more programs further include instructions for: performing at least one additional round of assaying when a number of positive samples P>D. In some embodiments, when exactly P positive samples are present in the plurality of samples, determining a number of positive pools comprises: if P≦D, identifying P*(D+1) positive pools; and if P>D, determining that (i) the determined positive pools include colliding samples, and (ii) at least one additional round of assaying is required. In some embodiments, each individual pool includes a tested set of samples distinct from each other individual pool. In some embodiments, the pooling scheme is a nonadaptive pooling scheme, such that samples are arranged in overlapping pools associated with the known pattern. In some embodiments, the decoding capability D is equal to 1. In some embodiments, the matrix has a size equal to: (R+1) by (R*(R+1))/2. In some embodiments, the number D is greater than 1. In some embodiments, the number D is constrained based on a quantity of sample material available. In some embodiments, the number R is constrained by the analytical sensitivity of the assaying. In some embodiments, the pooling scheme is utilized in the detection of Fragile X Syndrome. In some embodiments, assaying the pooled samples further comprises utilization of a capillary electrophoresis assay.

In some embodiments, the present invention includes a computer-implemented method of assaying a plurality of nucleic acid samples, the method comprising: generating a matrix of pools and samples using a pooling scheme, wherein the pooling scheme has a decoding capability equal to a number D; organizing the matrix by assigning one pool in a set of pools per row by one sample in a set of samples per column; assigning a number R of samples to each pool to create a known pattern of pools, wherein each sample in the set of pools is assigned a total number of D+1 times and any two pools have at most one sample in common; pooling the plurality of samples based at least in part on the pooling scheme and the matrix; assaying the pooled samples; in response to the assaying, determining a number of positive pools; identifying one or more positive samples based on the determined positive pools and the known pattern of pools; and displaying, on a display screen, the matrix as a visual pattern, the visual pattern representing each of the known pattern of pools, the identified one or more positive samples, and the determined positive pools.

### BRIEF DESCRIPTION OF THE DRAWINGS

FIG. 1A illustrates an exemplary group testing process for pooling and decoding a set of samples.

FIG. 1B illustrates an alternate representation of an exemplary group testing process for pooling and decoding a set of samples.

FIG. 2 illustrates an alternate representation of an exemplary group testing process for pooling and decoding a set of samples.

FIG. 3A illustrates an exemplary analysis for observing CGG repeat counts by semiautomatically identifying peaks in a fluorescence intensity trace.

FIG. 3B illustrates an exemplary analysis observing the sensitivity of a PCR-based FMR1 CGG repeat sizing.

FIG. 4A illustrates an exemplary simulation depicting a total number of group tests required based on an optimal sample batch size.

FIG. 4B illustrates an exemplary result of amortized cost savings based on an exemplary simulation.

FIG. 5 illustrates an exemplary summary of decoding results and depicting calls across a plurality of experiments.

FIG. 6 illustrates an exemplary process for determining a number of repeats of a nucleotide sequence in a gene according to various examples.

FIG. 7 illustrates log-scale histograms of allele size distribution by self-reported ethnicity.

FIG. 8 illustrates cumulative distributions of allele size by ethnicity.

FIG. 9 illustrates an exemplary computing system for determining a number of repeats of a nucleotide sequence in a gene.

### DETAILED DESCRIPTION

The present disclosure is directed to assaying a plurality of nucleic acid samples using a group testing scheme, and may be embodied as a system, method, or computer program product. Furthermore, the present invention may take the form of an entirely software embodiment, entirely hardware embodiment, or a combination of software and hardware embodiments. Even further, the present invention may take the form of a computer program product contained on a computer-readable storage medium, where computer-readable code is embodied on the storage medium. In another embodiment, the present invention may take the form of computer software implemented as a service (SaaS). Any appropriate storage medium may be utilized, such as optical storage, magnetic storage, hard disks, or CD-ROMs.

In the following description of the disclosure and examples, reference is made to the accompanying drawings in which it is shown by way of illustration specific examples that can be practiced. It is to be understood that other examples can be practiced and structural changes can be made without departing from the scope of the disclosure.

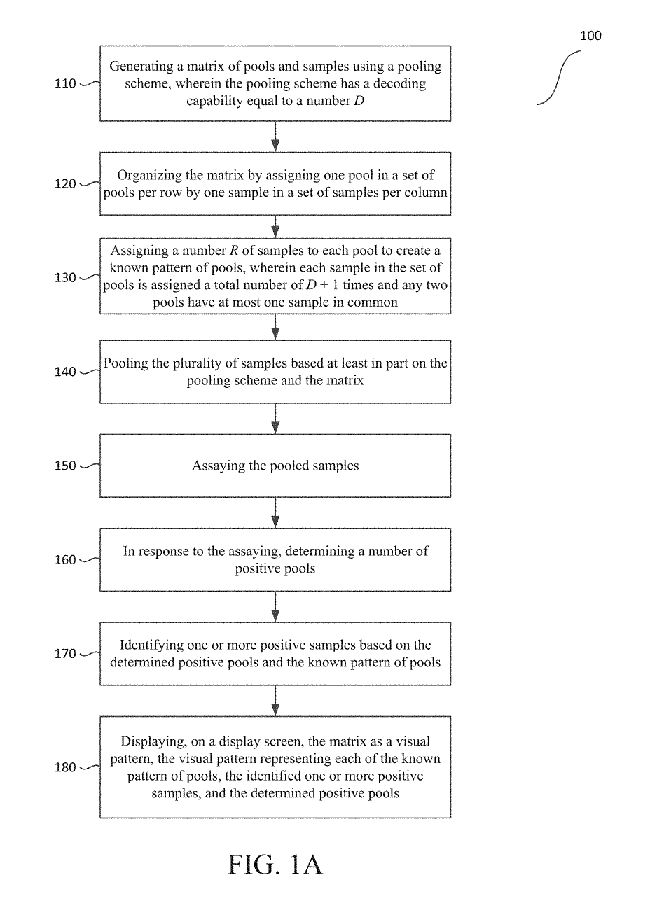

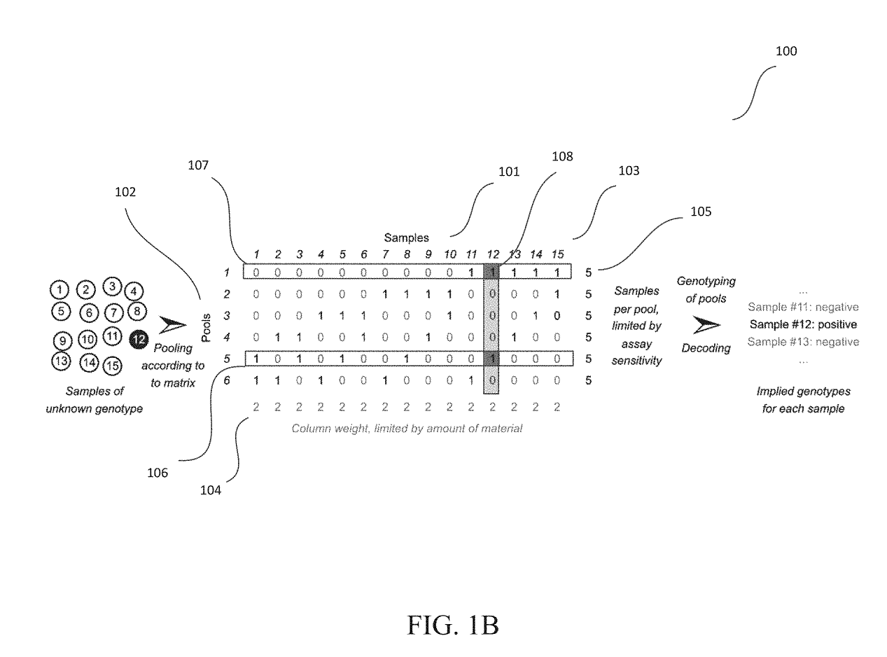

FIGS. 1A and 1B illustrate exemplary processes 100 for analyzing a plurality of nucleic acid samples. Nucleic acid samples may include, for example, deoxyribonucleic acid (DNA) samples or ribonucleic acid (RNA) samples. Group testing process 100 may, for example, be optimized for testing in which a small number of measurements of a combined set of samples (i.e., pools) are integrated to identify positive samples. Group testing process 100 may further be optimized for screening in testing involving rare abnormal genotypes. Group testing process 100 may further be optimized for screening in testing involving rare abnormal expansions and the analytical sensitivity of PCR-based sizing, for example. Regarding assay sensitivity, small, normal alleles may be amplified outside of a linear range of an assay, while amplification of larger alleles is improved, for example. The assay used with the methods described herein can include a capillary electrophoresis assay, such as the Fragile X assay described in Chen et al., An Information-Rich CGG Repeat Primed PCR That Detects the Full Range of Fragile X Expanded Alleles and Minimizes the Need for Southern Blot Analysis, Journal of Molecular Diagnostics (2010) vol. 12 (5) pp. 589-600, hereby incorporated by reference in its entirety. One skilled in the art will recognize that group testing process 100 may also be optimized for other processes and situations.

Methods of amplifying DNA from a DNA comprising a nucleic acid repeat region are known in the art, and have been reported in, for example, Chen et al. (id.); Alessandro Saluto et al., An Enhanced Polymerase Chain Reaction Assay to Detect Pre- and Full Mutation Alleles of the Fragile X Mental Retardation 1 Gene, Journal of Molecular Diagnostics (2005) vol. 7 (5) pp. 605-612; Feras M. Hantash et al., Qualitative assessment of FMR1 (CGG)n triplet repeat status in normal, intermediate, premutation, full mutation, and mosaic carriers in both sexes: Implications for Fragile X syndrome carrier and newborn screening, Genetics in Medicine (2010) 12:162-173; Stela Flilipovic-Sadic et al., A Novel FMR1 PCR Method for the Routine Detection of Low Abundance Expanded Alleles and Full Mutations in Fragile X Syndrome, Clinical Chemistry (2010) vol. 56 (3) pp. 399-408; and Flora Tassone et al., A rapid polymerase chain reaction-based screening method for identification of all expanded alleles of the Fragile X (FMR1) gene in newborn and high-risk populations, Journal of Molecular Diagnostics (2008) vol. 10(1) pp. 43-49. The content of each of the foregoing are incorporated by reference in their entirety. Methods of amplifying nucleic acid repeat regions are also described in, for example, U.S. Pat. No. 7,855,053, U.S. Pat. No. 8,409,805, and U.S. Patent Application Pub. No. 2010/0243451, the content of each of which is incorporated herein by reference in their entirety.

In one example, group testing process 100 may be configured to apply to both males and females, while further may be configured to determine the size of the CGG expansion and detect mosaicism. In another example, group testing process 100 may be configured to apply to one or more diseases including but not limited to myotonic dystrophy, Huntington disease, or spinocerebellar ataxias disease. In another example, group testing process 100 may be configured to apply to screening of other rare diseases where test volume and assay sensitivity are high.

A pooling scheme may, for example, determine how to carry out the group testing process 100. Referring to FIG. 1B, in an adaptive pooling scheme, samples 101 may first be pooled into nonoverlapping (i.e., distinct) groups. Furthermore, samples 101 in positive pools may be retested individually, or alternatively, may be retested recursively, such that samples 101 in nonpositive pools are not further tested, for example. As another example, a nonadpative scheme may be utilized, where samples may be combined into partially overlapping pools such that each positive sample 101 may create a known pattern of positive pools. In another example, testing a number R of samples in each pool may create a known pattern of pools. In yet another example, one or more positive samples may be identified based on the known pattern of pools and determined positive pools. In a nonadaptive scheme, patterns of pools may be decoded to identify positive samples with fewer tests than when testing each sample individually, for example.

A pooling scheme may further be configured to utilize an indicator matrix 103. For example, indicator matrix 103 may describe an assignment of a plurality of samples 101. In one example, samples 101 may be represented by a number N, and may correspond to the columns of the indicator matrix 103. The plurality of samples 101 may be further assigned to a plurality of pools 102. In another example, pools 102 may be represented by a number T, and may correspond to the rows of the indicator matrix 103. One or more positive pools may be identified by rows 106 and 107, where one or more positive samples may be identified by column 108 if no other samples are capable of creating the same pattern of positive pools. Although the system is described based on the row and column configuration above, indicator 103 matrix may further be transposed, such that pools 102 correspond to columns of indicator matrix 103, and samples 101 correspond to rows of indicator matrix 103.

Referring to FIG. 1A, an indicator matrix 103 may be generated at step 110, which includes generating a matrix of pools and samples using a pooling scheme, wherein the pooling scheme has a decoding capability equal to a number D. Furthermore, at step 120, process 100 is further configured for organizing the matrix by assigning one pool in a set of pools per row by one sample in a set of samples per column.

Referring back to FIG. 1B, indicator matrix 103 may further be configured to include a column weight 104 corresponding to a number of pools that a sample may be present in. As shown in FIG. 1B, column weight 104 may correspond to a value of “2.” However, those of skill in the art will appreciate that the column weight is not limited to the value of “2.” Furthermore, a column weight 104 may be constrained by a quantity of input material available. Indicator matrix 103 may further include a row weight 105 corresponding to a number of samples present in a pool. As shown in FIG. 1B, row weight 105 may correspond to a value of “5.” However, those of skill in the art will appreciate that the row weight 105 is not limited to the value of “5.” Furthermore, row weight 105 may be constrained by assay analytical sensitivity. In one example, a row weight 105 may be defined as R. Rₘₐₓ may be defined as when R is equal to the largest number of tested samples in a pool 102 such that the signal of a single positive sample is identified with substantial reliability. In one example, Rₘₐₓ may limit the implementation of compressed pooling schemes. In another example, a compressed pooling scheme may be a scheme such that the number of required pools scales as the logarithm of the number of samples. However, a compressed pooling scheme may be defined such that the number of required pools is defined by other properties. In another example, R is constant across all pools, in order to fully utilize resources and prevent the biasing of samples.

A pooling scheme may further include a decoding capability corresponding to a number D. In one example, a decoding capability corresponding to a number D equals zero may be associated with, for example, an adaptive pooling approach. In another example, decoding capability corresponding to a number D greater than zero may be associated with, for example, a non-adaptive pooling approach. Furthermore, where one round of sample pooling results in the determination of the identity of any number P of positive samples, and where P≦D, the decoding capability of the pooling approach may correspond to number D. In another example, a collision may occur where P>D, such that the decoding process fails. For example, when a decoding process fails, an additional assaying of samples may be required. In yet another example, the retested samples may be classified as “ambiguous samples.”

Referring back to FIG. 1A, process 100 is further configured, at step 130, for assigning a number R of samples in each pool to create a known pattern of pools, wherein each sample in the set of pools is assigned a total number of D+1 times and any two pools have at most one sample in common. Furthermore, process 100 is configured at step 140 to pool the plurality of samples based at least in part on the pooling scheme and an indicator matrix, and further is configured at step 150 to assay the pooled samples. In response to the assaying, at step 160 a determination is made of a number of positive pools. At step 170, process 100 is configured to identify one or more positive samples based on the determined positive pools and the known pattern of pools. Finally, at step 180, process 100 is configured to display, on a display screen, the matrix as a visual pattern, the visual pattern representing each of the known pattern of pools, the identified one or more positive samples, and the determined positive pools.

In another example, such as FXS screening, each size of allele may be distinguished from others by capillary electrophoresis, such that collisions may occur when alleles of the same size occur in more than D samples in a batch. A larger batch size may, in some instances, generally increase cost savings, but may result in a larger amount of collisions.

A pooling scheme may further result in assay cost savings. For example, assay cost savings may be determined based on the pooling scheme's amortized samples-to-tests ratio. A samples-to-test ratio may, for example, quantify the mean reduction in necessary assays relative to individual testing. In one example, if there are no collisions present, the samples-to-tests ratio may be represented as number N|number T.

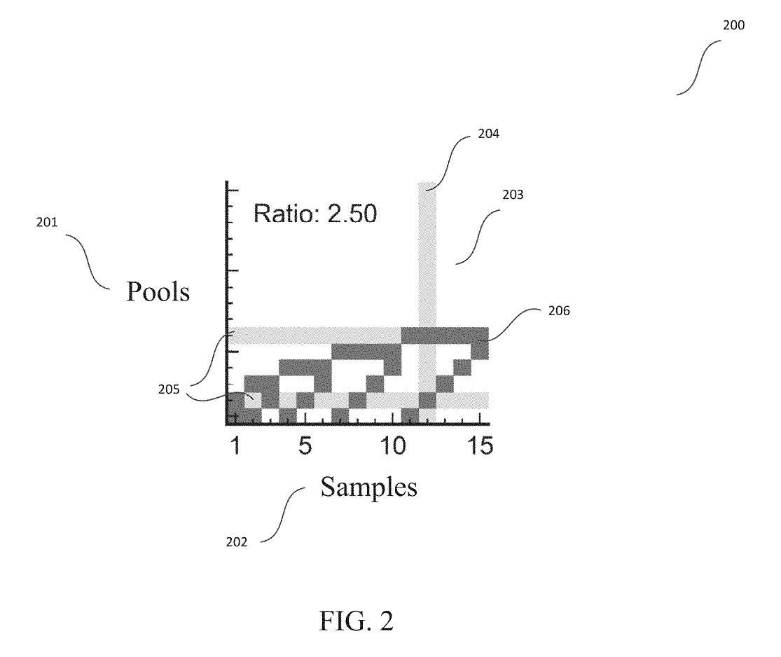

FIG. 2 illustrates an alternate representation of an exemplary group testing process 200. In one example, group testing process 200 may be configured to utilize a pooling scheme having a decoding capability of D=1, where the group testing process further utilizes an R value of 5 for each pool 201. Group testing process 200 may further be configured such that each sample 202 is present in exactly two pools 201, and such that any two pools may intersect only once. Furthermore, any two pools that contain a positive sample may identify the single positive in a batch of samples. As discussed above, Rₘₐₓ may be defined where R is equal to the largest number of tested samples in a pool 102 such that the signal of a single positive sample is identified with substantial reliability. In one example, group testing process 200 may result in the generation of a matrix 203 having a size equal to (Rₘₐₓ+1) by (Rₘₐₓ*(Rₘₐₓ+1))/2.

In another example, Python code may be used to generate group testing process 200 represented by matrix 203, and the pooling scheme used to develop group testing process 200 may be referred to as a “Staircase” (SC) scheme. Those skilled in the art will also recognize that software other than Python may be used to generate the matrix. In another example, matrix 203 may represent pools as rows and samples as columns, and may further represent the identified one or more positive samples with highlighted column(s) 204 and the determined positive pools with highlighted row(s) 205. In another example, matrix 203 may exhibit a recursive structure 206, and thus may be depicted as a recognizable visual pattern on a display screen. For example, the visual pattern may include specific “1s” or “0s” within matrix 203, such that the resultant matrix appearance resembles a pattern such as a “Staircase” or other recursive-type pattern. In another example, the visual pattern may correspond to other recognizable patterns, such as slanted lines, straight lines, diagonal lines, or a mosaic pattern resembling any combination of lines and dots. The display of such a visual pattern to a user on a display is advantageous in that the pattern may allow a user to easily detect pools that contain a positive sample, and further, identify the positive sample based on the determined positive pools. For example, in the “Staircase” recursive pattern depicted in FIG. 2, identification of a positive sample is further improved by permitting a user to easily identify the positive sample by locating positive pools within each of a horizontal section and a diagonal section of the pattern, and thus, locating the positive sample by determining the sample common to each pool.

Furthermore, exemplary group testing process 200 may be configured to utilize a recursive pattern of pooling. A given pooling scheme may be configured to be optimized when a specific number of samples N is provided in an overall layout, and a number of samples R are tested together in a pool. For example, a SC scheme may be optimized when 210 samples are to be tested and 20 samples are tested together. In general, the larger the number of samples in an overall layout, the greater probability that there are one or more positive samples in a batch. Furthermore, a decoding algorithm for a SC scheme may be configured as a lookup table process when two positive pools unambiguously identify a sample. In another example, where there are more than two positive pools, the m-choose-2 combinations of pairs of m positive pools may identify a potential positive sample, and the result may be ambiguous. For instance, in one example, two true positive samples may cause four pools to be positive, which in turn may cause six samples to be identified as ambiguous. Such a scenario may necessitate a retest of the six ambiguous samples.

Furthermore, a theoretical cost savings ratio of the SC scheme may be represented as Rₘₐₓ/2, when there are either zero or one positive samples in a batch of samples. In one example, Rₘₐₓ/2 may be configured as a best theoretical ratio for the matrix 203 discussed above, where matrix 203 has a decoding capability of D=1, and where group testing process 200 is configured such that each sample 202 is present in exactly two pools 201.

As another example, a modified SC scheme may be utilized. Under a modified SC scheme, Rₘₐₓ may be defined where R is equal to the largest number of tested samples in a pool such that the signal of a single positive sample is identified with substantial reliability. Furthermore, the modified SC scheme may include a decoding capability of D>1, such that one sample is present in exactly D+1 pools. In yet another example, Rₘₐₓ in a modified SC scheme is proportional to D. In one embodiment, Rₘₐₓ may be optimized to produce a best theoretical cost savings by selecting an appropriate number of pools and an appropriate number of samples to be tested. For example, when (i) any two pools have at most one sample in common, and (ii) each sample is present in exactly D+1 pools, a best theoretical cost savings may be achieved when

$${R}_{max} = \frac{( D + 1 ) ⋆ N}{T} ,$$

wherein N represents a number of samples to be tested, T represents a number of sample pools, and Rₘₐₓ is a whole number. In one embodiment, N and T may be varied in order to achieve a best theoretical cost savings. For example, for D=2, group testing process may produce optimized results where Rₘₐₓ equals 3, 7, 15, 31, 63, etc.

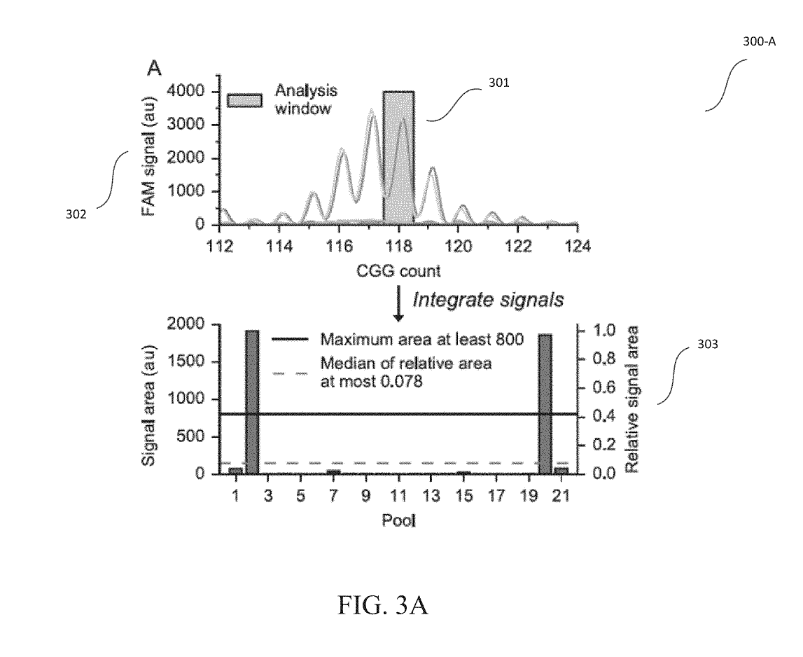

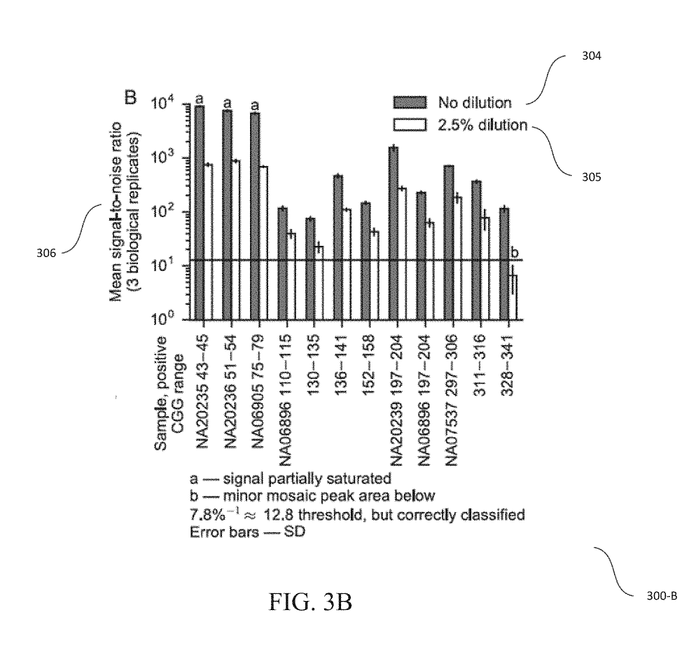

FIG. 3A illustrates an exemplary analysis 300-A for observing CGG repeat counts by semiautomatically identifying peaks in a fluorescense intensity trace. In one example, a fluorescence intensity area may be integrated over an analysis window 301 defined by one CGG repeat for all assayed pools. In another example, the presence of a large signal may be recognized by identifying a maximum area of any pool which is identified as above a given signal value 302 (e.g., a value of 800). Furthermore, the presence of a large signal-to-noise ratio may be recognized by identifying a median signal value which is smaller than a specific signal value 302 (e.g., a value of 0.078). If, for example, identification is made of a large signal and a large signal-to-noise ratio, then, all pools with a specific normalized area 303 above a specific signal value 302 (e.g., 0.2), which are within analysis window 301, may be determined positive for the given CGG repeat size of analysis window 301. In one example, the specific signal values 302 may be determined by maximizing the harmonic mean of precision and recall in at least one independent experiment. In yet another example, the specific normalized area 303 may be determined heuristically. Furthermore, those skilled in the art will appreciate that specific signal values 301 and specific normalized area 303 may be determined according to other means.

FIG. 3B illustrates an exemplary analysis 300-B for the sensitivity of a PCR-based FMR1 CGG repeat sizing. For example, samples 301 may be configured to be assayed independently. In one example, an independent assay 304 corresponds to an assay with no dilution. Furthermore, for example, samples 302 may be configured to be assayed in a background of 39 normal alleles. In another example, a background of 39 normal alleles may correspond to an assay 305 with 2.5% dilution. At a 2.5% dilution level, for example, a single X chromosome may be represented in a pool of 40 chromosomes, which may correspond to a worst case example of an all-female 20-member pool. Furthermore, a mean pool with 20 individuals may be expected to contain only 30× chromosomes. Based on exemplary analysis 300-B, the major variants of the CGG expansion may be detected with a high confidence. Even further, a signal-to-noise ratio 306 may be calculated, for example, by dividing a mean positive signal area by a median non-positive signal area.

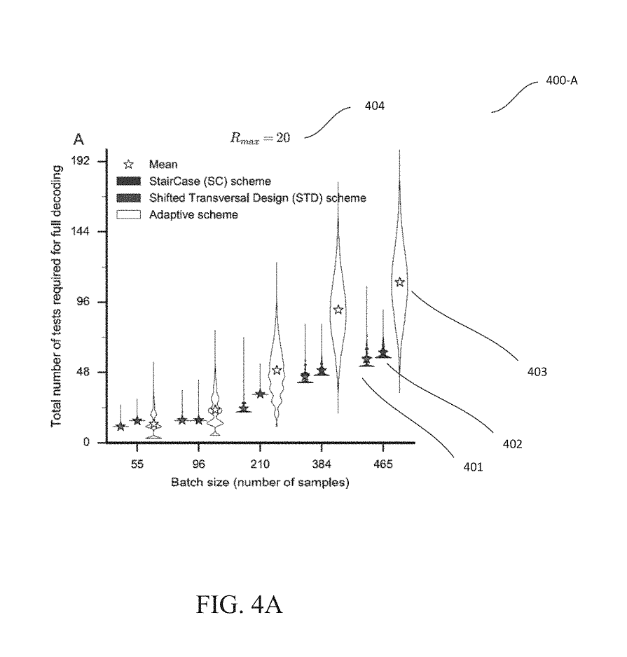

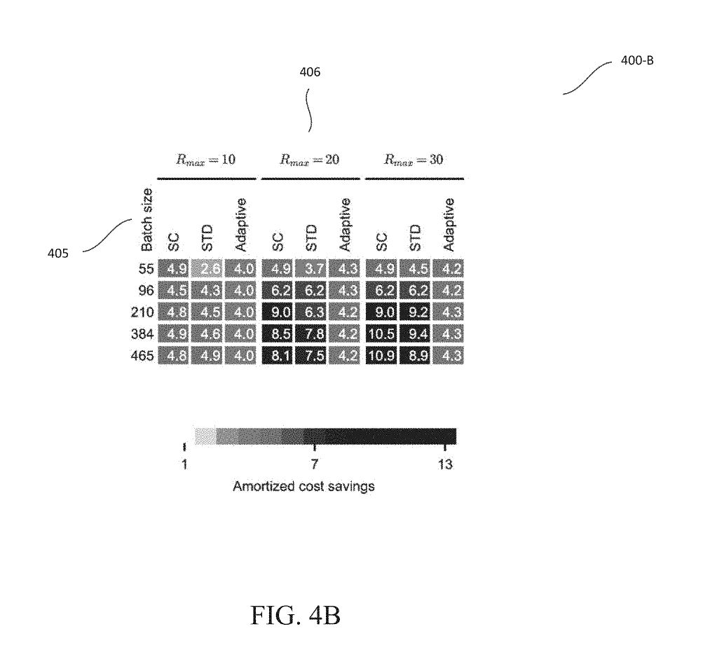

FIG. 4A illustrates an exemplary simulation 400-A depicting a total number of group tests required based on an optimal sample batch size. Exemplary simulation 400-A may depict a frequency of abnormal FMR1 alleles using different pooling schemes. As depicted in FIG. 4A, results for three pooling scheme simulations are presented, such as a SC scheme 401, a “Shifted Transversal Design” (STD) scheme 402, and an “adaptive scheme” 403. For example, STD scheme 402 may correspond to the STD scheme as described in Thierry-Mieg, referred to above and incorporated herein by reference. In one example, each pooling scheme may be simulated with an Rₘₐₓ value 404 equal to 20. In another example, SC scheme 401 may be configured to detect an identity up to one positive sample per batch in one iteration of sample pooling. In another example, adaptive scheme 403 may be configured to detect one positive sample per batch in more than one iteration of testing, and may further be configured to pool samples without overlap and recursively test positive pools by splitting those pools in half.

Furthermore, an adaptive scheme 403 may be associated with a decoding capability of zero, and may require retesting of pools if a nonnormal allele is observed. SC scheme 401 may only require limited retesting if a nonnormal allele is observed, such that fewer samples are retested than in adaptive scheme 403. As another example, where a collision occurs in the SC scheme 401, ambiguous samples may be configured to be simulated as being retested individually. In the case of a simulation on a suboptimal batch size, a simulation may be configured to utilize a smaller optimal scheme using nonoverlapping subsets of the batch. In one example, simulations may be configured to evaluate performance of a plurality of pooling schemes by analyzing randomized batches of samples. An amortized cost savings may further be calculated by dividing a batch size by a mean number of tests required for complete decoding. In another example, SC scheme 401 may be configured to be simulated by utilizing an individual retest of ambiguous samples.

FIG. 4B illustrates an exemplary result of amortized cost savings based on exemplary simulation 400-B. In one example, cost-savings ratios may be improved based on increasing an Rₘₐₓ value 404 and increasing a sample batch size. In another example, chance of collision may increase based on increasing an Rₘₐₓ value 404 and increasing a sample batch size. In another example, cost-savings ratios may decrease based on increasing an Rₘₐₓ value 404 and increasing a sample batch size. A specific batch size 405 may be chosen for a specific Rₘₐₓ value 406 in order to optimize performance and compatibility with assay sensitivity. Furthermore, utilization of a SC pooling scheme in the context of trinucleotide repeat expansion disorder testing may offer greater than 10-fold reduction in assay costs over single-plex methods.

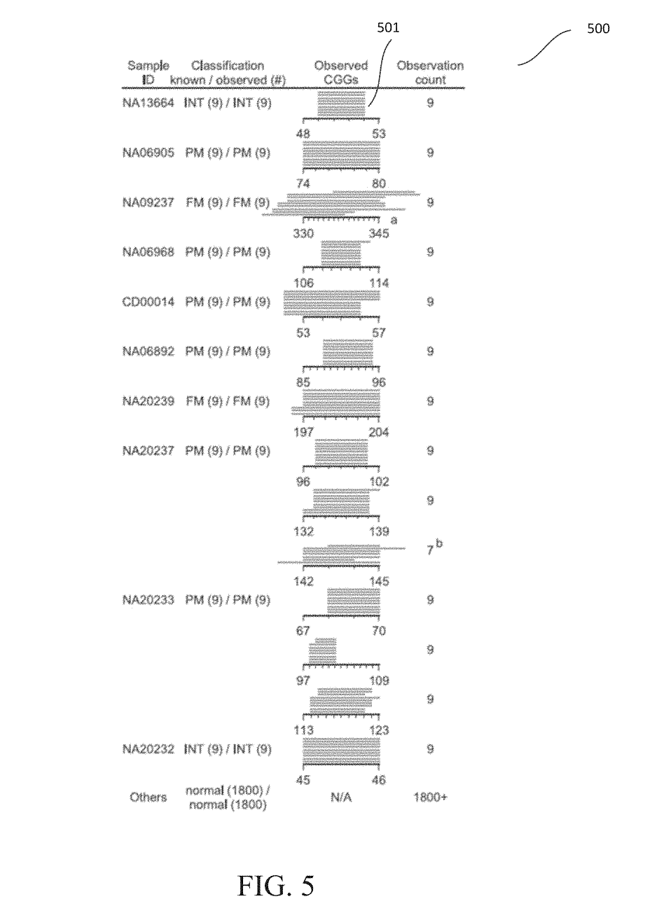

FIG. 5 illustrates an exemplary summary 500 of decoding results, depicting calls across a plurality of experiments. Stacked lines 501 may depict a width of each peak determined to be associated with a given sample over three assay replicates of three pooling scheme iterations. In one example, exemplary summary 500 may be achieved using three variants of a 210-sample pooling scheme with an Rₘₐₓ value equal to 20. In another example, each variant may differ only by an order of samples in a pooling matrix, and thus, may differ only by the order of sample composition of each pool. In yet another example, exemplary summary 500 may be achieved by differing assignment of samples to pools, and thus differing the number of positive samples that are contained in the same pool.

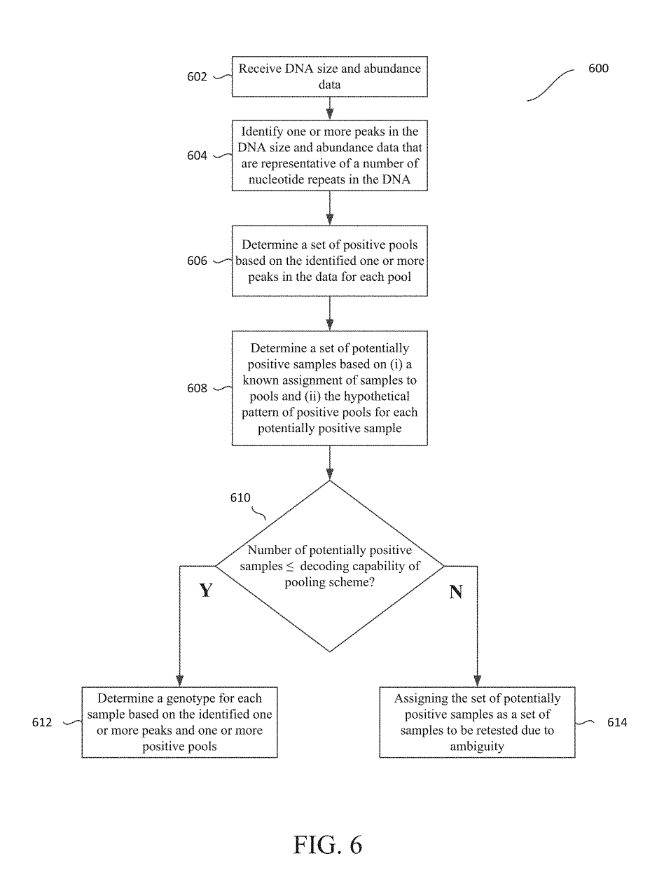

FIG. 6 illustrates an exemplary process 600 for determining a number of nucleotide repeats in a gene according to various examples. In one example, the determining a number of nucleotide repeats in a gene includes utilization of an assay for determining the existence of Fragile X Syndrome. Process 600 will be described herein as determining a number of CGG repeats in a DNA sample comprising a CGG-rich region. However, it should be appreciated that process 600 may similarly be used to determine a number of any desired nucleotide sequence in any desired gene to identify any type of nucleic acid repeat disorder.

At block 602, DNA size and abundance data may be received by one or more processors of a computing device. The size and abundance data may be generated by resolving DNA amplification products using capillary electrophoresis (e.g., to produce an electropherogram) or the like. The DNA amplification products may be generated from the DNA using a primer set including a first primer recognizing a region outside of the CGG-rich region, and a second primer recognizing a region outside of the CGG-rich region that is on a side opposite the region recognized by the first primer. It should be appreciated that other genes may be represented by the DNA size and abundance data.

In some examples, the DNA size and abundance data may include multiple data points having a fluorescence value and an associated time at which the data point sample was taken. In these examples, the DNA size and abundance data may be transformed from the time domain to a base-pair length domain. This may be accomplished using a DNA ladder having fragments of known length and by converting the DNA size and abundance data x-value from machine sample time to base-pair length. In some examples, the DNA fragments corresponding to the individual's DNA may be labeled by a fluorescent dye, such as FAM, and the fragments corresponding to the DNA ladder may be labeled by a distinct fluorescent dye, such as ROX. In some examples, high FAM signal intensity may create crosstalk between fluorescent detection channels, adding spurious peaks or removing true ones and impeding automation detection of ROX ladder peaks. In these instances, a prior distribution on expected locations of ladder peaks may be used to match observed peaks to the prior using dynamic programming to simultaneously assign peaks and minimize the squared-deviation in peak location using the following formula:

$$min\_dev ( i , p ) = min [ {{min}_{q - 1}}^{p - 1} ( min\_dev ( i - 1 , q ) + penalty ( i , p ) ) , {{min}_{q = 1}}^{p - 1} ( min\_dev ( i - 2 , q ) + penalty ( i , p ) ) + MISSED\_PEAK \_PENALTY ] i indexes fragment sizes , p indexes peaks$$

In some examples, the sampling interval of the machine used to generate the DNA size and abundance data may not be linear in base-pair length. In these examples, once the DNA size and abundance data is converted into the base-pair length domain, the DNA size and abundance data may be interpolated using linear interpolation, cubic spline interpolation, or zero-order hold/nearest neighbor interpolation, and sampled to a constant resolution. Any desired resolution may be used and, in one example, a sampling frequency of four samples per base-pair may be used. The result of the sampling may be a set of data or a signal representative of the full-length amplicon (e.g., of the 5′ UTR of the FMR1 gene). The component is expected to have a long period or may not be periodic since the DNA size and abundance data is expected to include only one or a small number of full-length amplicons, depending on sample zygosity, that are unlikely to be separated by only one repeat.

Referring back to process 600 shown in FIG. 6, at block 604, one or more peaks in the DNA size and abundance data that are representative of a number of nucleotide repeats in the DNA may be identified. To identify the peaks, each signal or set of data, represented by the function ƒ, may be interpolated using a cubic spline and the interpolated data may be used to approximate the first derivative ƒ′ and the second derivative ƒ″ of the signal or set of data ƒ. Next, a root C of the first derivative ƒ′ that also satisfies the condition that the second derivative at C ƒ″ (C)<0 may be identified. This root C may be designated as the center of the corresponding peak. Values L and R may be the locations of roots of ƒ′ that are adjacent (e.g., closest roots of ƒ′ that have higher and lower CGG repeat counts) to the left and right, respectively, of root C. To compute the peak boundaries L′ and R′ for the peak centered at C, the following equations may be used:

L′=min xε[L,C]s.t.|ƒ′(x)|>D

R′=max xε[C,R]s.t.|ƒ′(x)|>D

In other words, the left peak boundary L′ may be the smallest X-axis value (e.g., CGG repeat count) between adjacent root L and center C that has a first derivative ƒ′ whose absolute value is greater than a cutoff D. The value of D may depend on the dynamic range of the DNA size and abundance data (and thus, on the sample protocol and hardware) and may be selected to be a value corresponding to the location that a human would identify as the peak boundary. Similarly, the right peak boundary R′ may be the largest X-axis value (e.g., CGG repeat count) between center C and adjacent root R that has a first derivative ƒ′ whose absolute value is greater than a cutoff D. This peak identification process may be performed for each root C of the first derivative ƒ′ of each signal or set of data that also satisfies the condition that the second derivative at C ƒ″ (C)<0. While a specific peak detection algorithm is described above, it should be appreciated that other peak detection algorithms may be used.

Once the set of peaks in the data are identified, the peaks in each set may be filtered to remove peaks that have a high probability of being noise, rather than ones accurately reflecting the full-length amplicon. In some examples, the peak filtering may include identifying thin peaks whose widths are less than a first threshold number of CGG repeats (e.g., 1.5) and whose heights are less than a machine-dependent second threshold. The exact values of these first and second thresholds may be determined and set empirically or through calculations to remove peaks resulting from noise. The identified thin peaks may be removed from their respective set of peaks, or may otherwise be identified (e.g., using a flag) as being noise. Peaks having heights that are smaller than the height of a peak immediately to their right (e.g., having larger CGG repeat counts) that are within the same set of data may also be removed from their respective set of peaks or may otherwise be identified as being noise since it is expected that the height of each peak is to be less than the previous peak (e.g., to the left) due to the decreasing efficiency of amplification with increasing length.

In some examples, some peaks may be merged if it is determined that one or more of the peaks are attributable to noise. The merging of peaks may include treating the two or more merged peaks as a single peak, meaning that the largest peak of the merged peaks may be treated as the true peak. In some examples, peaks within each set of data having peaks above a threshold number (e.g., 55) of repeats may be merged if they are within a threshold number (e.g., 10) of repeats of each other. All peaks, regardless of repeat count, within the same set of data may be merged if they are within a threshold number of repeats (e.g., 5) and more than a factor of 2 different in amplitude.

Referring back to process 600 of FIG. 6, a set of positive pools is determined at step 606. For example, determination of the positive pools may be based on the identified one or more peaks in the data for each pool. At step 608, a set of potentially positive samples is determined based at least in part on (i) a known assignment of samples to pools and (ii) a hypothetical pattern of positive pools for each potentially positive sample. Furthermore, at step 610, a determination is made whether a number of potentially positive samples is equal to, or less than, a decoding capability of a respective pooling scheme. In one example, when a number of potentially positive samples is equal to, or less than, a decoding capability of a respective pooling scheme, then at step 612 a genotype is determined for each sample based on the identified one or more peaks and the one or more positive pools. In another example, when a number of potentially positive samples greater than a decoding capability of a respective pooling scheme, then at step 614 the set of potentially positive samples is assigned as a set of samples to be retested due to ambiguity.

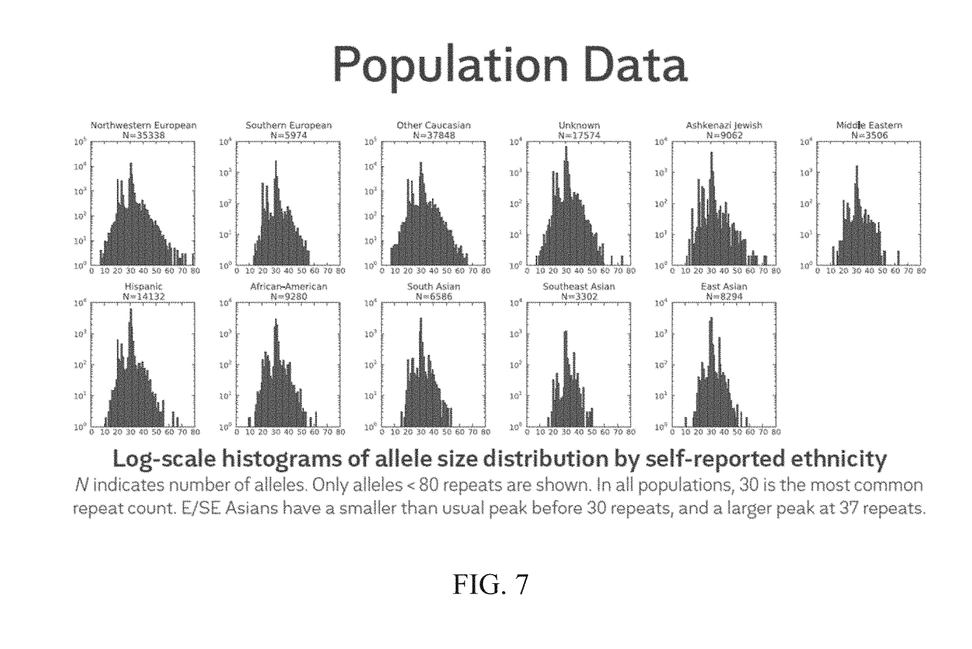

FIG. 7 shows log-scale histograms of allele size distribution by self-reported ethnicity. N indicates the number of alleles. Only alleles <80 repeats are shown. In all populations, 30 is the most common repeat count. East and Southeast Asians have a smaller than usual peak before 30 repeats, and a larger peak at 37 repeats. For the purposes of pooled testing, each allele size may be treated as a separate positive, such that the likelihood of colliding samples is determined by the frequency of each allele size, rather than the frequency of having an abnormal repeat size. Furthermore, in some embodiments, samples associated with the same or similar ethnicity may be distributed across pooling experiments such that the ethnicity composition of any given pooling experiment is diverse. In one example, testing samples of diverse ethnicity in a pooling experiment may result in a reduction in the chance of sample genotype collisions, and therefore, may result in a reduction of the number of sample retests required. In another example, other attributes may be used to assign samples to pooling experiments. Such characteristics may include, but are not limited to, those which render a sample more likely to be a carrier of an expansion (e.g., family history), or more likely to be a carrier of an allele that is also present in the same experiment (e.g., relatedness between samples). In another example, samples known to carry expansions may be excluded from pooling experiments, and may further be assayed individually.

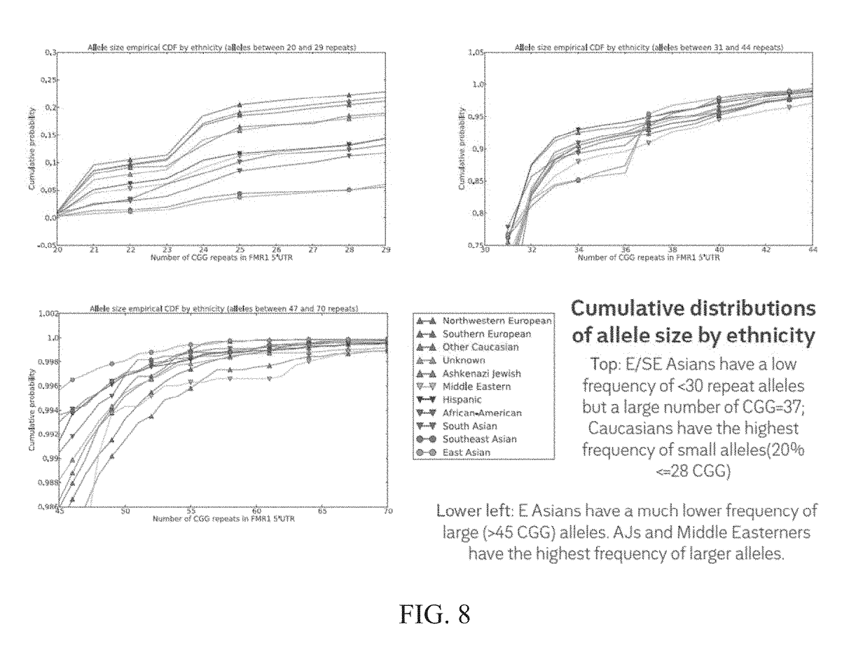

FIG. 8 illustrates a worldwide catalog of Fragile X allele sizes. As shown in FIG. 8, East Asians tend to have shorter alleles and Middle Easterners longer ones, but other groups are not significantly differentiated for intermediate or larger alleles. Automated signal processing for pooled PCR+CE-based testing for Fragile X syndrome is efficient and reliable, allowing cost-effective population-scale carrier screening. FMR1 repeat lengths vary significantly by ethnicity: East and Southeast Asians have very low probabilities of both small (<30) and large (>45) alleles. East and Southeast Asians have a large number of CGG-37 alleles. Caucasians have the highest frequency of small alleles (20%<=28 CGG). Samples with reported Middle Eastern or Ashkenazi Jewish heritage showed a higher probability of alleles >45 repeats.

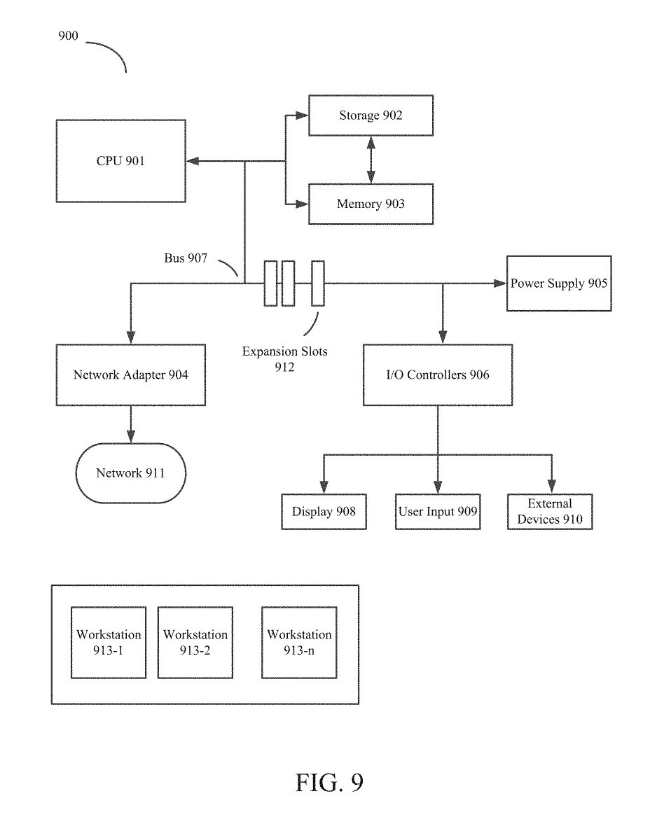

FIG. 9 illustrates a general purpose computing system 900 in which one or more systems, as described herein, may be implemented. System 900 may include, but is not limited to known components such as central processing unit (CPU) 901, storage 902, memory 903, network adapter 904, power supply 905, input/output (I/O) controllers 906, electrical bus 907, one or more displays 908, one or more user input devices 909, and other external devices 910. It will be understood by those skilled in the art that system 900 may contain other well-known components which may be added, for example, via expansion slots 912, or by any other method known to those skilled in the art. Such components may include, but are not limited, to hardware redundancy components (e.g., dual power supplies or data backup units), cooling components (e.g., fans or water-based cooling systems), additional memory and processing hardware, and the like.

System 900 may be, for example, in the form of a client-server computer capable of connecting to and/or facilitating the operation of a plurality of workstations or similar computer systems over a network. In another embodiment, system 900 may connect to one or more workstations over an intranet or internet network, and thus facilitate communication with a larger number of workstations or similar computer systems. Even further, system 900 may include, for example, a main workstation or main general purpose computer to permit a user to interact directly with a central server. Alternatively, the user may interact with system 900 via one or more remote or local workstations 913. As will be appreciated by one of ordinary skill in the art, there may be any practical number of remote workstations for communicating with system 900.

CPU 901 may include one or more processors, for example Intel® Core™ i7 processors, AMD FX™ Series processors, or other processors as will be understood by those skilled in the art. CPU 901 may further communicate with an operating system, such as Windows NT® operating system by Microsoft Corporation, Linux operating system, or a Unix-like operating system. However, one of ordinary skill in the art will appreciate that similar operating systems may also be utilized. Storage 902 may include one or more types of storage, as is known to one of ordinary skill in the art, such as a hard disk drive (HDD), solid state drive (SSD), hybrid drives, and the like. In one example, storage 902 is utilized to persistently retain data for long-term storage. Memory 903 may include one or more types of memory as is known to one of ordinary skill in the art, such as random access memory (RAM), read-only memory (ROM), hard disk or tape, optical memory, or removable hard disk drive. Memory 903 may be utilized for short-term memory access, such as, for example, loading software applications or handling temporary system processes.

As will be appreciated by one of ordinary skill in the art, storage 902 and/or memory 903 may store one or more computer software programs. Such computer software programs may include logic, code, and/or other instructions to enable processor 901 to perform the tasks, operations, and other functions as described herein, and additional tasks and functions as would be appreciated by one of ordinary skill in the art. Operating system 902 may further function in cooperation with firmware, as is well known in the art, to enable processor 901 to coordinate and execute various functions and computer software programs as described herein. Such firmware may reside within storage 902 and/or memory 903.

Moreover, I/O controllers 906 may include one or more devices for receiving, transmitting, processing, and/or interpreting information from an external source, as is known by one of ordinary skill in the art. In one embodiment, I/O controllers 906 may include functionality to facilitate connection to one or more user devices 909, such as one or more keyboards, mice, microphones, trackpads, touchpads, or the like. For example, I/O controllers 906 may include a serial bus controller, universal serial bus (USB) controller, FireWire controller, and the like, for connection to any appropriate user device. I/O controllers 906 may also permit communication with one or more wireless devices via technology such as, for example, near-field communication (NFC) or Bluetooth™. In one embodiment, I/O controllers 906 may include circuitry or other functionality for connection to other external devices 910 such as modem cards, network interface cards, sound cards, printing devices, external display devices, or the like. Furthermore, I/O controllers 906 may include controllers for a variety of display devices 908 known to those of ordinary skill in the art. Such display devices may convey information visually to a user or users in the form of pixels, and such pixels may be logically arranged on a display device in order to permit a user to perceive information rendered on the display device. Such display devices may be in the form of a touch-screen device, traditional non-touch screen display device, or any other form of display device as will be appreciated be one of ordinary skill in the art.

Furthermore, CPU 901 may further communicate with I/O controllers 906 for rendering a graphical user interface (GUI) on, for example, one or more display devices 908. In one example, CPU 901 may access storage 902 and/or memory 903 to execute one or more software programs and/or components to allow a user to interact with the system as described herein. In one embodiment, a GUI as described herein includes one or more icons or other graphical elements with which a user may interact and perform various functions. For example, GUI 907 may be displayed on a touch screen display device 908, whereby the user interacts with the GUI via the touch screen by physically contacting the screen with, for example, the user's fingers. As another example, GUI may be displayed on a traditional non-touch display, whereby the user interacts with the GUI via keyboard, mouse, and other conventional I/O components 909. GUI may reside in storage 902 and/or memory 903, at least in part as a set of software instructions, as will be appreciated by one of ordinary skill in the art. Moreover, the GUI is not limited to the methods of interaction as described above, as one of ordinary skill in the art may appreciate any variety of means for interacting with a GUI, such as voice-based or other disability-based methods of interaction with a computing system.

Moreover, network adapter 904 may permit device 900 to communicate with network 911. Network adapter 904 may be a network interface controller, such as a network adapter, network interface card, LAN adapter, or the like. As will be appreciated by one of ordinary skill in the art, network adapter 904 may permit communication with one or more networks 911, such as, for example, a local area network (LAN), metropolitan area network (MAN), wide area network (WAN), cloud network (IAN), or the Internet.

One or more workstations 913 may include, for example, known components such as a CPU, storage, memory, network adapter, power supply, I/O controllers, electrical bus, one or more displays, one or more user input devices, and other external devices. Such components may be the same, similar, or comparable to those described with respect to system 900 above. It will be understood by those skilled in the art that one or more workstations 913 may contain other well-known components, including but not limited to hardware redundancy components, cooling components, additional memory/processing hardware, and the like.

As used herein, the terminology as used throughout the description of the invention is for the purpose of describing particular embodiments only. Such terminology does not limit the scope of the invention in any way. For example, singular forms of “a,” “an” and “the” are intended to include plural forms unless indicated otherwise. Furthermore, terms such as “comprises” or “comprising” specify the presence of indicated features, components, steps, etc., but do not preclude the presence or addition of one or more other features, components, steps, etc. The description may also include the term “in,” which may include “in” and “on” unless clearly indicated otherwise. Furthermore, usage of the term “or” includes both conjunctive and disjunctive meanings, unless clearly indicated otherwise. That is, unless expressly stated otherwise, the term “or” may include “and/or.”

It will be further understood that various modifications to the invention may be made by one skilled in the art without departing from the spirit and scope of the invention as defined in the claims. For example, numerous changes, substitutions, and variations with respect to the systems and methods as described may occur. One of ordinary skill in the art will understand that various alternative embodiments may be employed to practice the invention, and that any feature may be combined with any other feature, whether such features are preferred or not.

Although the disclosure and examples have been fully described with reference to the accompanying drawings, it is to be noted that various changes and modifications will become apparent to those skilled in the art. Such changes and modifications are to be understood as being included within the scope of the disclosure and examples as defined by the appended claims.

What is claimed is:

1. A method for assaying a plurality of nucleic acid samples, the method comprising:
generating a matrix of pools and samples using a pooling scheme, wherein the pooling scheme has a decoding capability equal to a number D;
organizing the matrix by assigning one pool in a set of pools per row by one sample in a set of samples per column;
assigning a number R of samples to each pool to create a known pattern of pools, wherein each sample in the set of pools is assigned a total number of D+1 times and any two pools have at most one sample in common;
pooling the plurality of samples based at least in part on the pooling scheme and the matrix;
assaying the pooled samples;
in response to the assaying, determining a number of positive pools;
identifying one or more positive samples based on the determined positive pools and the known pattern of pools; and
displaying, on a display screen, the matrix as a visual pattern, the visual pattern representing each of the known pattern of pools, the identified one or more positive samples, and the determined positive pools.

2. The method of claim 1, wherein a number of positive samples P is determined in a single iteration of analysis when P≦D.

3. The method of claim 1, further comprising:
performing at least one additional round of assaying when a number of positive samples P>D.

4. The method of claim 1, wherein when exactly P positive samples are present in the plurality of samples, determining a number of positive pools comprises:
if P≦D, identifying P*(D+1) positive pools; and
if P>D, determining that (i) the determined positive pools include colliding samples, and (ii) at least one additional round of assaying is required.

5. The method of claim 1, wherein each individual pool includes a tested set of samples distinct from each other individual pool.

6. The method of claim 1, wherein the pooling scheme is a nonadaptive pooling scheme, such that samples are arranged in overlapping pools associated with the known pattern.

7. The method of claim 1, wherein the decoding capability D is equal to 1.

8. The method of claim 1, wherein the matrix has a size equal to:
(R+1) by (R*(R+1))/2.

9. The method of claim 1, wherein the number D is greater than 1.

10. The method of claim 1, wherein the number D is constrained based on a quantity of sample material available.

11. The method of claim 1, wherein the number R is constrained by the analytical sensitivity of the assaying.

12. The method of claim 1, wherein the pooling scheme is utilized in the detection of Fragile X Syndrome.

13. The method of claim 1, wherein assaying the pooled samples further comprises utilization of a capillary electrophoresis assay.

14. A system for assaying a plurality of nucleic acid samples, the system comprising:
a display;
one or more processors; and
a memory storing one or more programs, wherein the one or more programs include instructions configured to be executed by the one or more processors, causing the one or more processors to perform operations comprising:
generating a matrix of pools and samples using a pooling scheme, wherein the pooling scheme has a decoding capability equal to a number D;
organizing the matrix by assigning one pool in a set of pools per row by one sample in a set of samples per column;
assigning a number R of samples to each pool to create a known pattern of pools, wherein each sample in the set of pools is assigned a total number of D+1 times and any two pools have at most one sample in common;
pooling the plurality of samples based at least in part on the pooling scheme and the matrix;
assaying the pooled samples;
in response to the assaying, determining a number of positive pools;
identifying one or more positive samples based on the determined positive pools and the known pattern of pools; and
displaying, on a display screen, the matrix as a visual pattern, the visual pattern representing each of the known pattern of pools, the identified one or more positive samples, and the determined positive pools.

15. The system of claim 14, wherein a number of positive samples P is determined in a single iteration of analysis when P≦D.

16. The system of claim 14, wherein the one or more programs further include instructions for:
performing at least one additional round of assaying when a number of positive samples P>D.

17. The system of claim 14, wherein when exactly P positive samples are present in the plurality of samples, determining a number of positive pools comprises:
if P≦D, identifying P*(D+1) positive pools; and
if P>D, determining that (i) the determined positive pools include colliding samples, and (ii) at least one additional round of assaying is required.

18. The system of claim 14, wherein each individual pool includes a tested set of samples distinct from each other individual pool.

19. The system of claim 14, wherein the pooling scheme is a nonadaptive pooling scheme, such that samples are arranged in overlapping pools associated with the known pattern.

20. The system of claim 14, wherein the decoding capability D is equal to 1.

21. The system of claim 14, wherein the matrix has a size equal to:
(R+1) by (R*(R+1))/2.

22. The system of claim 14, wherein the number D is greater than 1.

23. The system of claim 14, wherein the number D is constrained based on a quantity of sample material available.

24. The system of claim 14, wherein the number R is constrained by the analytical sensitivity of the assaying.

25. The system of claim 14, wherein the pooling scheme is utilized in the detection of Fragile X Syndrome.

26. The system of claim 14, wherein assaying the pooled samples further comprises utilization of a capillary electrophoresis assay.

27. A non-transitory computer readable storage medium having instructions stored thereon, the instructions, when executed by one or more processors, cause the processors to perform operations for assaying a plurality of nucleic acid samples, the operations comprising:
generating a matrix of pools and samples using a pooling scheme, wherein the pooling scheme has a decoding capability equal to a number D;
organizing the matrix by assigning one pool in a set of pools per row by one sample in a set of samples per column;
assigning a number R of samples to each pool to create a known pattern of pools, wherein each sample in the set of pools is assigned a total number of D+1 times and any two pools have at most one sample in common;
pooling the plurality of samples based at least in part on the pooling scheme and the matrix;
assaying the pooled samples;
in response to the assaying, determining a number of positive pools;
identifying one or more positive samples based on the determined positive pools and the known pattern of pools; and
displaying, on a display screen, the matrix as a visual pattern, the visual pattern representing each of the known pattern of pools, the identified one or more positive samples, and the determined positive pools.

28. The storage medium of claim 27, wherein a number of positive samples P is determined in a single iteration of analysis when P≦D.

29. The storage medium of claim 27, wherein the one or more programs further include instructions for:
performing at least one additional round of assaying when a number of positive samples P>D.

30. The storage medium of claim 27, wherein when exactly P positive samples are present in the plurality of samples, determining a number of positive pools comprises:
if P≦D, identifying P*(D+1) positive pools; and
if P>D, determining that (i) the determined positive pools include colliding samples, and (ii) at least one additional round of assaying is required.

31. The storage medium of claim 27, wherein each individual pool includes a tested set of samples distinct from each other individual pool.

32. The storage medium of claim 27, wherein the pooling scheme is a nonadaptive pooling scheme, such that samples are arranged in overlapping pools associated with the known pattern.

33. The storage medium of claim 27, wherein the decoding capability D is equal to 1.

34. The storage medium of claim 27, wherein the matrix has a size equal to:
(R+1) by (R*(R+1))/2.

35. The storage medium of claim 27, wherein the number D is greater than 1.

36. The storage medium of claim 27, wherein the number D is constrained based on a quantity of sample material available.

37. The storage medium of claim 27, wherein the number R is constrained by the analytical sensitivity of the assaying.

38. The storage medium of claim 27, wherein the pooling scheme is utilized in the detection of Fragile X Syndrome.

39. The storage medium of claim 27, wherein assaying the pooled samples further comprises utilization of a capillary electrophoresis assay.

40. A computer-implemented method of assaying a plurality of nucleic acid samples, the method comprising:
generating a matrix of pools and samples using a pooling scheme, wherein the pooling scheme has a decoding capability equal to a number D;
organizing the matrix by assigning one pool in a set of pools per row by one sample in a set of samples per column;
assigning a number R of samples to each pool to create a known pattern of pools, wherein each sample in the set of pools is assigned a total number of D+1 times and any two pools have at most one sample in common;
pooling the plurality of samples based at least in part on the pooling scheme and the matrix;
assaying the pooled samples;
in response to the assaying, determining a number of positive pools;
identifying one or more positive samples based on the determined positive pools and the known pattern of pools; and
displaying, on a display screen, the matrix as a visual pattern, the visual pattern representing each of the known pattern of pools, the identified one or more positive samples, and the determined positive pools.
